(19) United States
(12) Patent Application Publication
(10) Pub. No.: US 2023/0364039 A1
(43) Pub. Date: Nov. 16, 2023

(54) Methods of Managing Side Effects of a Vasopressin Receptor Antagonist Therapy

(71) Applicant: Emory University, Atlanta, GA (US)

(72) Inventors: Jeff Sands, Atlanta, GA (US); Janet Klein, Decatur, GA (US)

(21) Appl. No.: 18/030,491

(22) PCT Filed: Oct. 14, 2021
(86) PCT No.: PCT/US2021/055003

§ 371 Date: Apr. 5, 2023

Related U.S. Application Data
(60) Provisional application No. 63/091,334, filed on Oct. 14, 2020.

(51) Int. Cl. A61K 31/16; A61K 31/55; A61P 13/00

(52) U.S. Cl. CPC A61K 31/16; A61K 31/55; A61P 13/00

(57) ABSTRACT

This disclosure relates to methods of managing polyuria as a side effect of vasopressin receptor antagonist therapies. In certain embodiments, this disclosure relates to methods of treating or preventing polyuria as a side effect of a vasopressin receptor antagonist therapy comprising administering a vasopressin receptor antagonist to a human subject in combination with an effective amount of an activator of adenosine monophosphate activated protein kinase (AMPK). In certain embodiments, the subject is diagnosed with autosomal dominant polycystic kidney disease (ADPKD). In certain embodiments, the subject is diagnosed with polyuria.

### CROSS-REFERENCE TO RELATED APPLICATIONS

This application claims the benefit of U.S. Provisional Application No. 63/091,334 filed Oct. 14, 2020. The entirety of this application is hereby incorporated by reference for all purposes.

### BACKGROUND

Vasopressin receptor antagonists are reported to treat hyponatremia, i.e., a decrease in serum sodium concentrations. Hyponatremia is sometimes associated with renal disease. The vasopressin V2-receptor antagonist, tolvaptan is indicated to slow the progression of cyst development and renal insufficiency in adults at risk of progressing autosomal dominant polycystic kidney disease (ADPKD). One side effect of tolvaptan therapy is the production of abnormally large volumes of dilute urine, polyuria. Thus, there is a need to identify methods of managing polyuria during tolvaptan therapy.
Efe et al. report metformin improves urine concentration in rodents with nephrogenic diabetes insipidus. JCI Insight. 2016, 1(11): e88409.
See also Sands et al., Physiological insights into novel therapies for nephrogenic diabetes insipidus, Am J Physiol Renal Physiol, 311: F1149-F1152, 2016; U.S. Pat. Nos. 9,827,222 and 8,623,897, US Pub. Pat. Appl. No. 2016/0367516; and Int. Pat. WO2018/144570.

References cited herein are not an admission of prior art.

### SUMMARY

This disclosure relates to methods of managing polyuria as a side effect of vasopressin receptor antagonist therapies. In certain embodiments, this disclosure relates to methods of treating or preventing polyuria as a side effect of a vasopressin receptor antagonist therapy comprising administering a vasopressin receptor antagonist to a human subject in combination with an effective amount of an activator of adenosine monophosphate activated protein kinase (AMPK). In certain embodiments, the subject is diagnosed with autosomal dominant polycystic kidney disease (ADPKD). In certain embodiments, the subject is diagnosed with polyuria.

In certain embodiments, the vasopressin receptor antagonist is selected from N-(4-(7-chloro-5-hydroxy-2,3,4,5-tetrahydro-1H-benzo[b]azepine-1-carbonyl)-3-methylphenyl)-2-methylbenzamide (tolvaptan), conivaptan, lixivaptan, and satavaptan, prodrug, derivative, or salt thereof.

In certain embodiments, this disclosure relates to methods of treating or preventing polyuria as a side effect of a vasopressin receptor antagonist therapy comprising administering a vasopressin receptor antagonist such as N-(4-(7-chloro-5-hydroxy-2,3,4,5-tetrahydro-1H-benzo[b]azepine-1-carbonyl)-3-methylphenyl)-2-methylbenzamide (tolvaptan) to a human subject in combination with an effective amount of 1,1′-(dodecane-1,12-diyl)bis(cyclopropane-1-carboxamide)(NP-5033), prodrug, derivative, or salt thereof.

In certain embodiments, this disclosure relates to methods of treating or preventing hyponatremia related to a renal disease, cirrhosis, congestive heart failure (CHF), cancer, or a syndrome of inappropriate antidiuretic hormone (SIADH) and preventing polyuria as a side effect of a vasopressin receptor antagonist therapy comprising administering a vasopressin receptor antagonist such as N-(4-(7-chloro-5-hydroxy-2,3,4,5-tetrahydro-1H-benzo[b]azepine-1-carbonyl)-3-methylphenyl)-2-methylbenzamide (tolvaptan) to a human subject in combination with an effective amount of 1,1′-(dodecane-1,12-diyl)bis(cyclopropane-1-carboxamide)(NP-5033), prodrug, derivative, or salt thereof.

In certain embodiments, this disclosure relates to pharmaceutical compositions comprising a vasopressin receptor antagonist and an activator of adenosine monophosphate activated protein kinase (AMPK).

### BRIEF DESCRIPTION OF THE SEVERAL VIEWS OF THE DRAWINGS

FIG. 1 shows data indicating urine osmolality increases with AMPK activator treatment. Tolvaptan (10 mg/kg/day) was delivered by gavage feeding daily. AMPK activator was given at 2.5, 5 and 10 mg/kg/day for up to 14 total days, with daily urine collection for osmolality determination. Light bars: Osmolality in rats receiving tolvaptan only. Black bars: treated rats. Mean±s.e., n=4-5 for lower doses, 8-10 for 10 mg/kg/day, *=p<0.05.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Prior to describing the various embodiments, the following definitions are provided and should be used unless otherwise indicated. Further, headings provided herein are for convenience only and do not interpret the scope or meaning of the claims.

The compounds of the present disclosure may form one or more salts, tautomers, solvates, or contain one or more chiral centers and exist in different optically active forms. When the compound contains one chiral center, the compound comprises an enantiomer. The present disclosure includes mixtures of salts, stereoisomers, enantiomers, diastereomers, tautomers, or solvates. Enantiomers can be resolved by methods known in the art, such as crystallization, chiral chromatography and the like. When the compound contains more than one chiral center, diastereomers may be present. The present disclosure includes specific optically pure isomers which have been resolved, as well as mixtures of diastereomers. Diastereomers can be resolved by methods known in the art, such as crystallization and preparative chromatography.

Unless the context requires otherwise, throughout the specification and claims which follow, the word “comprise” and variations thereof, such as, “comprises,” “comprising” “including,” “containing,” or “characterized by,” are to be construed in an open, inclusive sense, that is, as “including, but not limited to” and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

The terms “administer,” “administering” or “administration” as used herein refer to either directly administering a compound (also referred to as an agent of interest) or pharmaceutically acceptable salt of the compound (agent of interest) or a composition to a subject.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, adding a hydroxyl group, replacing an oxygen atom with a sulfur atom, or replacing an amino group with a hydroxyl group, oxidizing a hydroxyl group to a carbonyl group, reducing a carbonyl group to a hydroxyl group, and reducing a carbon-to-carbon double bond to an alkyl group or oxidizing a carbon-to-carbon single bond to a double bond. A derivative optional has one or more substitutions. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

As used herein, the term “prodrug” refers a compound that, after administration, is metabolized (i.e., converted within the body) into a pharmacologically active drug. Examples include amides and alkyl esters of carboxylic acids such as ethyl esters, isopropyl esters, n-butyl esters, aryl esters, (acyloxy)alkyl esters, [(alkoxycarbonyl)oxy]methyl esters, (oxodioxolyl)methyl esters or amino acid esters, or alkoxy esters of hydroxyl groups such as, acetate esters, benzoate esters, alkyl ethers, amino acids esters, glycolic acid esters, malic acid esters, acyloxyalkyl esters, alkoxycarbonyloxy alkyl esters, S-acylthioalkyl esters, hydroxylamine amides, phosphonylmethoxy ethers, phosphates, phosphoramidates, and combinations thereof.

The phrase “pharmaceutically acceptable” is employed herein to refer to those agents of interest/compounds, salts, compositions, dosage forms, etc, which are within the scope of sound medical judgment suitable for use in contact with the tissues of human beings and/or other mammals without excessive toxicity, irritation, allergic response, or other problem or complication, commensurate with a reasonable benefit/risk ratio. In some respects, pharmaceutically acceptable means approved by a regulatory agency of the federal or a state government or listed in the U.S. Pharmacopeia or other generally recognized pharmacopeia for use in mammals (e.g., animals), and more particularly, in humans.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The composition may be administered to patients in an amount effective, especially to enhance pharmacological response in an animal or human organism. As used herein, the term “effective amount” refers to an amount sufficient to realize a desired biological effect. The appropriate dosage may vary depending upon known factors such as the pharmacodynamic characteristics of the particular active agent, age, health, and weight of the host organism; the condition(s) to be treated, nature and extent of symptoms, kind of concurrent treatment, frequency of treatment, the need for prevention or therapy and/or the effect desired. The dosage will also be calculated dependent upon the particular route of administration selected. Further refinement of the calculations necessary to determine the appropriate dosage for treatment is routinely made by a practitioner, in the light of the relevant circumstances. The titer may be determined by conventional techniques.

Vasopressin receptor antagonists are agents that specifically bind to vasopressin receptors (V1A, V1B and V2) and block the action of vasopressin (antidiuretic hormone, ADH), which is a hormone released by the pituitary gland. Examples include tolvaptan, conivaptan, lixivaptan, and satavaptan.

### AMPK Activators

As used herein, the term “AMPK activator” refers to a substance that activates adenosine monophosphate kinase, AMPK.

In certain embodiments, the AMPK activator is 1,1′-(dodecane-1,12-diyl)bis(cyclopropane-1-carboxamide)(NP-5033), derivative, prodrug, or salt thereof.

In embodiments, the AMPK activator is a compound of Formula I,

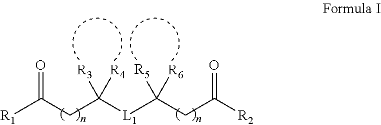

derivative, prodrug, or salt thereof, wherein R₁ and R₂ are independently selected from a group consisting of hydroxy, alkoxy, NHR′ wherein R′ is hydrogen, alkyl, cycloalkyl, SO₂R or COR, wherein R is selected from alkyl or cycloalkyl; R₃ and R₄ or R₅ and R₆ are independently selected from hydrogen, or alkyl; R₃ and R₄ and/or R₅ and R₆ may form a cyclic ring of 3-5 carbon atoms; n is independently selected from 0 or 1; Li is independently a linear aliphatic chain optionally containing from 10 to 14 carbon atoms. In embodiments, at least one of R₃ and R₄ and/or R₅ and R₆ forms a cyclic ring of 3-5 carbon atoms, R₃ and R₄ or R₅ and R₆.

In embodiments, the compound of formula I are compounds of formula IA,

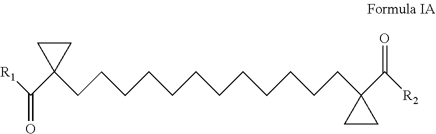

derivative, prodrug, or salt thereof, wherein R₁ and R₂ are independently selected from a group consisting of hydroxyl, alkoxy, or NHR′ wherein R′ is hydrogen, alkyl, or cycloalkyl.

In embodiments, the compound of Formula I is 1,1′-(dodecane-1,12-diyl)bis(cyclopropane-1-carboxamide)(NP-5033) or derivatives or salts thereof.

In embodiments, the compound of Formula I is 1,1′-(decane-1,10-diyl)bis(cyclopropane-1-carboxamide) or derivatives or salts thereof.

In embodiments, the compound of Formula I is 1,1′-(dodecane-1,12-diyl)bis(cyclopropane-1-carboxylic acid) derivative, prodrug, or salt thereof.

In embodiments, the compound of Formula I is 1,1′-(decane-1,10-diyl)bis(cyclopropane-1-carboxylic acid) derivative, prodrug, or salt thereof.

In embodiments, the compound of Formula I is 1-(12-(1-(cyclopropylcarbamoyl)cyclopropyl)dodecyl)cyclopropane-1-carboxylic derivative, prodrug, or salt thereof.

In embodiments, the compound of Formula I is 1-(10-(1-(cyclopropylcarbamoyl)cyclopropyl)decyl)cyclopropane-1-carboxylic derivative, prodrug, or salt thereof.

In certain embodiments, a compound of Formula I include the following examples. The preparation of these examples are provided for in US Patent Publications 20140121267 and 20120071528.

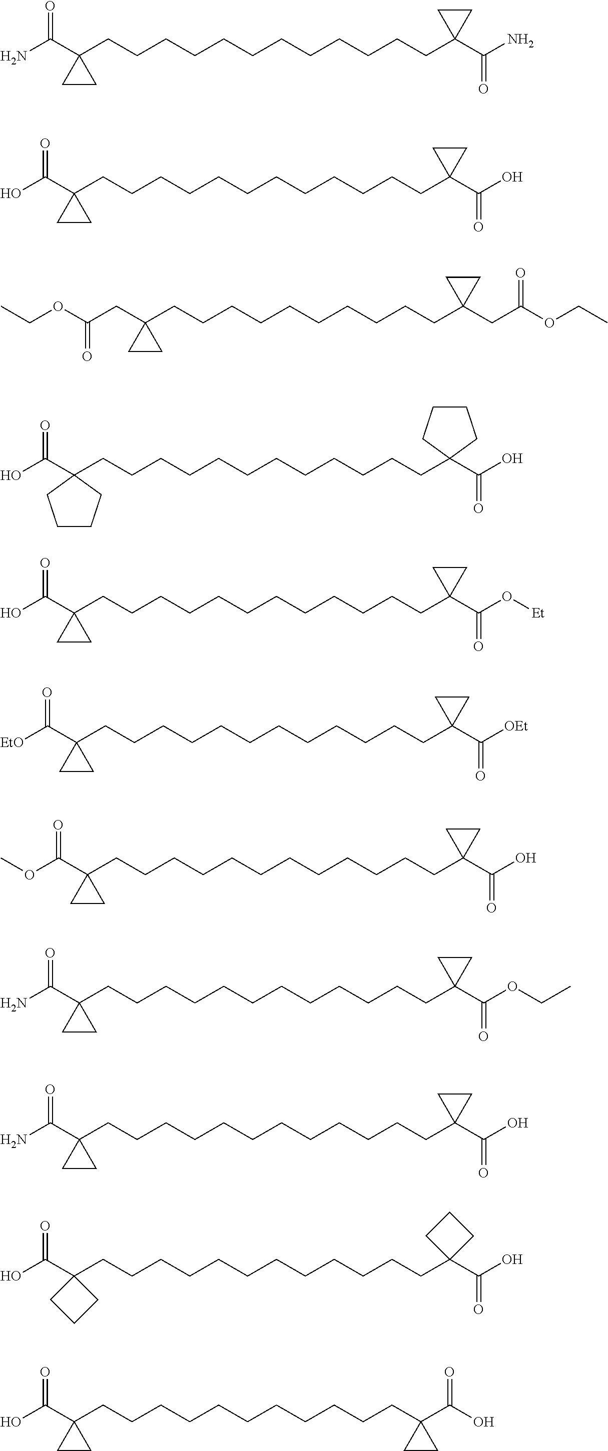

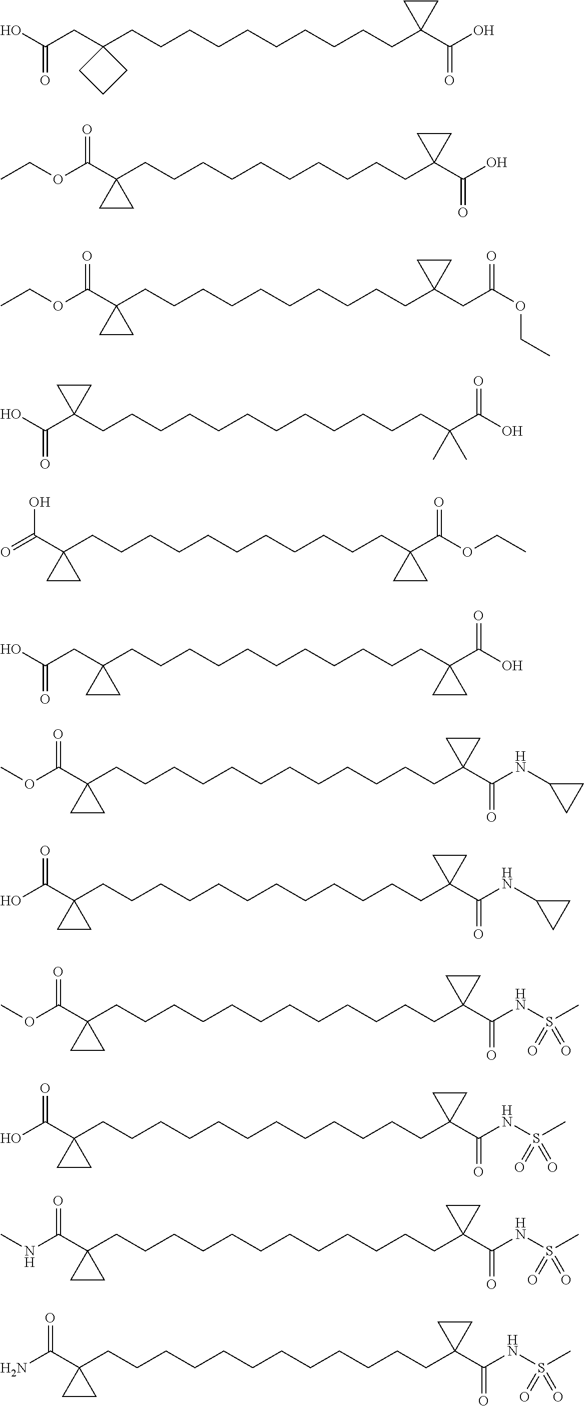

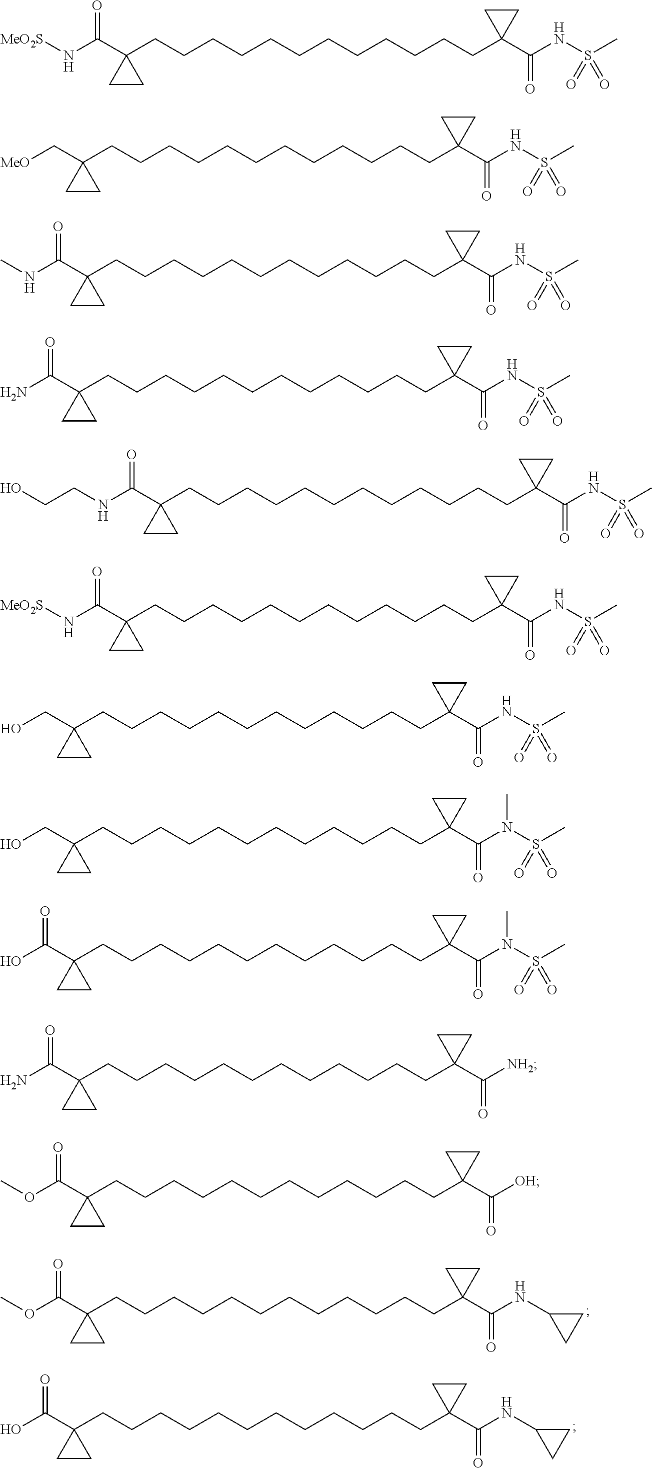

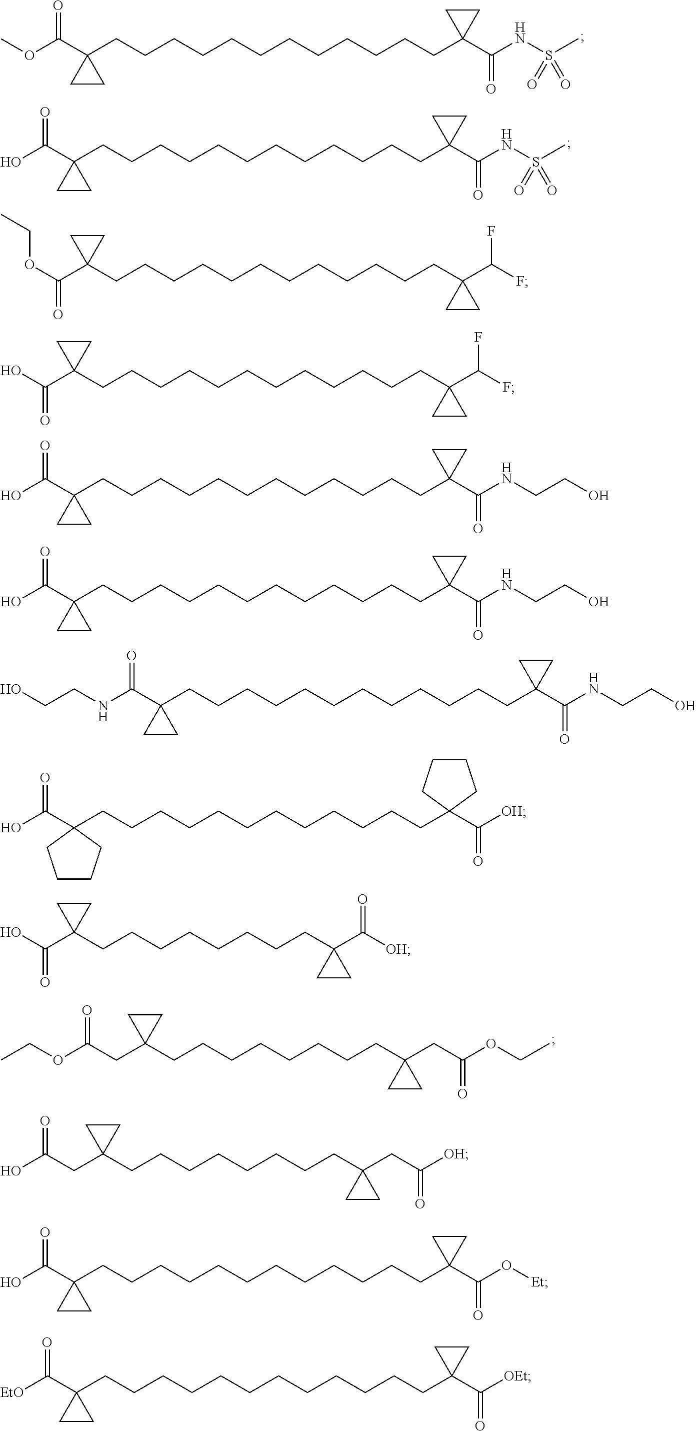

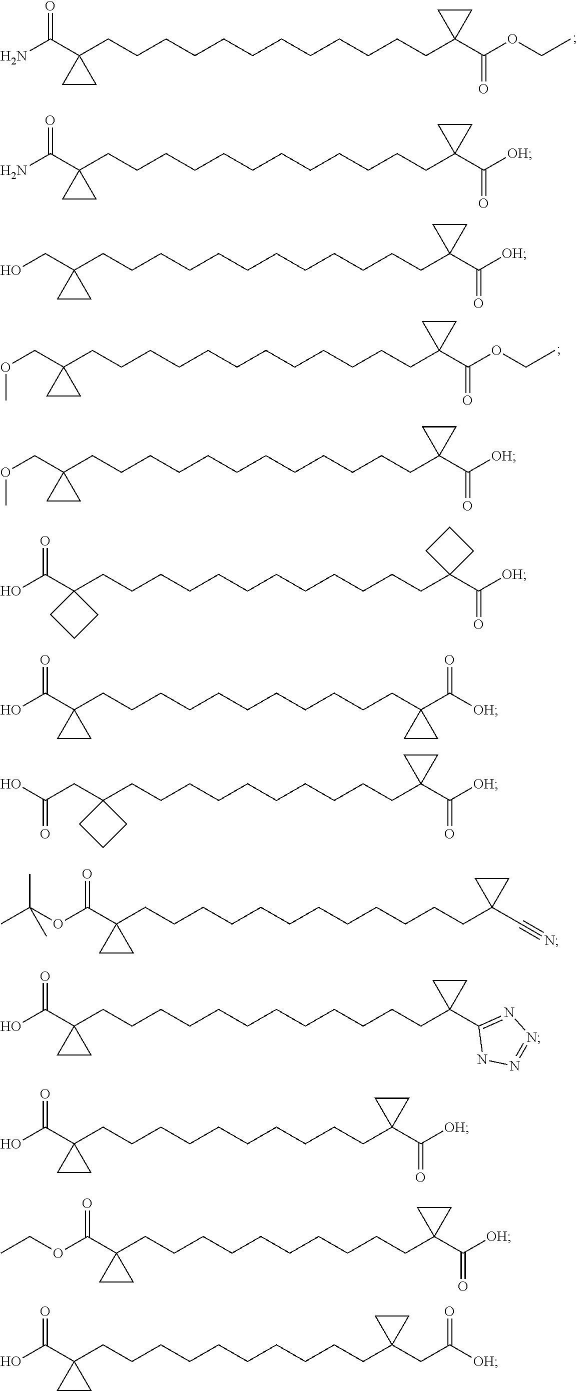

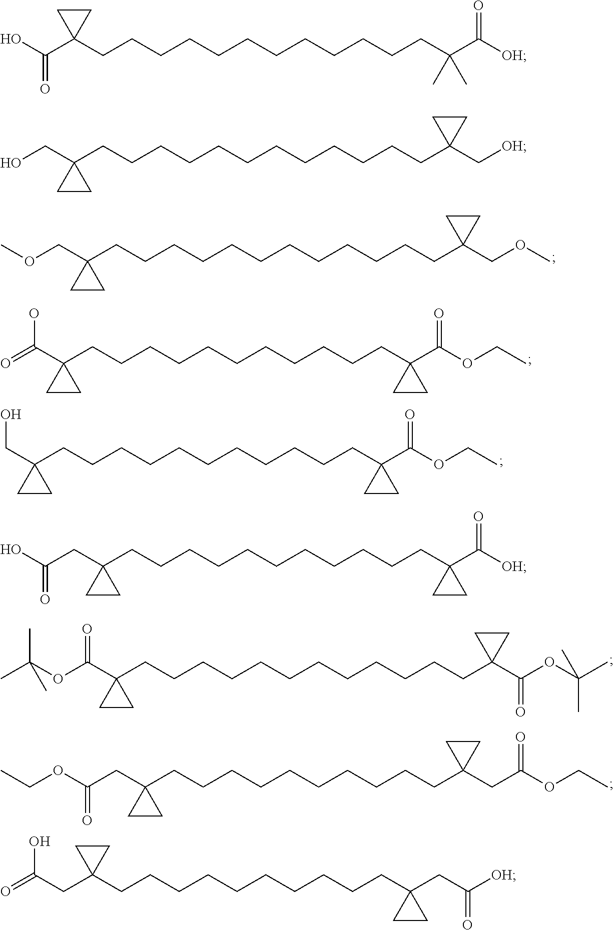

In certain embodiments, the AMPK activator is metformin derivative, prodrug, or salt thereof.

In certain embodiments, the AMPK activator is 5-aminoimidazole-4-carboxamide-1-beta-D-ribofuranoside derivative, prodrug, or salt thereof.

In certain embodiments, the AMPK activator is 4-hydroxy-3-(2′-hydroxy-[1,1′-biphenyl]-4-yl)-6-oxo-6,7-dihydrothieno[2,3-b]pyridine-5-carbonitrile derivative, prodrug, or salt thereof.

In certain embodiments, the AMPK activator is 5-(3-(4-(2-(4-Fluorophenyl)ethoxy)phenyl)propyl)furan-2-carboxylic acid derivative, prodrug, or salt thereof.

In certain embodiments, the AMPK activator is O2′,O3′,O5′-tri-acetyl-N6-(3-hydroxylaniline)adenosine derivative, prodrug, or salt thereof.

In certain embodiments, the AMPK activator is 2-chloro-5-[[5-[[5-(4,5-dimethyl-2-nitrophenyl)-2-furanyl]methylene]-4,5-dihydro-4-ox-o-2-thiazolyl]amino]benzoic acid derivative, prodrug, or salt thereof.

### Methods of Use

This disclosure relates to methods of managing polyuria as a side effect of vasopressin receptor antagonist therapies. In certain embodiments, this disclosure relates to methods of treating or preventing polyuria as a side effect of a vasopressin receptor antagonist therapy comprising administering a vasopressin receptor antagonist to a human subject in combination with an effective amount of an activator of adenosine monophosphate activated protein kinase (AMPK).

In certain embodiments, this disclosure relates to methods of improving urine concentrating ability of a subject in a subject receiving a vasopressin receptor antagonist therapy comprising administering a vasopressin receptor antagonist to a human subject in combination with an effective amount of an activator of adenosine monophosphate activated protein kinase (AMPK).

In certain embodiments, the subject is diagnosed with a renal disease or disfunction such as a result of autosomal dominant polycystic kidney disease (ADPKD), chronic kidney failure, or associated with heart failure, liver disease, cancer, lung cancer, or a syndrome of inappropriate antidiuretic hormone secretion (SIADH). In certain embodiments, the subject is diagnosed with polyuria.

In certain embodiments, the subject is diagnosed with autosomal dominant polycystic kidney disease (ADPKD) due to a mutation in one of the following genes: PKD1, PKD2, GANAB and DNAJB11.

In certain embodiments, this disclosure relates to methods of treating or preventing hyponatremia (<136 mmol/L) or hypernatremia (>145 mmol/L) a subject in a subject receiving a vasopressin receptor antagonist therapy comprising administering a vasopressin receptor antagonist to a human subject in combination with an effective amount of an activator of adenosine monophosphate activated protein kinase (AMPK).

In certain embodiments, the vasopressin receptor antagonist is selected from N-(4-(7-chloro-5-hydroxy-2,3,4,5-tetrahydro-1H-benzo[b]azepine-1-carbonyl)-3-methylphenyl)-2-methylbenzamide (tolvaptan), conivaptan, lixivaptan, and satavaptan, prodrug, derivative, or salt thereof.

In certain embodiments, this disclosure relates to methods of treating or preventing polyuria as a side effect of a vasopressin receptor antagonist therapy comprising administering N-(4-(7-chloro-5-hydroxy-2,3,4,5-tetrahydro-1H-benzo[b]azepine-1-carbonyl)-3-methylphenyl)-2-methylbenzamide (tolvaptan) to a human subject in combination with an effective amount of 1,1′-(dodecane-1,12-diyl)bis(cyclopropane-1-carboxamide)(NP-5033), derivative, prodrug, or salt thereof.

In certain embodiments, this disclosure relates to methods disclosed herein wherein a vasopressin receptor antagonist therapy comprises administering N-(4-(7-chloro-5-hydroxy-2,3,4,5-tetrahydro-1H-benzo[b]azepine-1-carbonyl)-3-methylphenyl)-2-methylbenzamide (tolvaptan) to a human subject in combination with an effective amount of 1,1′-(dodecane-1,12-diyl)bis(cyclopropane-1-carboxamide)(NP-5033), derivative, prodrug, or salt thereof.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 60-100 mg orally per day and 1,1′-(dodecane-1,12-diyl)bis(cyclopropane-1-carboxamide)(NP-5033), derivative, prodrug, or salt thereof is administered between 10 and 3,000 mg per day or more.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 60-100 mg orally per day and 1,1′-(dodecane-1,12-diyl)bis(cyclopropane-1-carboxamide)(NP-5033), derivative, prodrug, or salt thereof is administered between 10 and 1,000 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 60-100 mg orally per day and 1,1′-(dodecane-1,12-diyl)bis(cyclopropane-1-carboxamide)(NP-5033), derivative, prodrug, or salt thereof is administered between 200 and 1,000 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 60-100 mg orally per day and 1,1′-(dodecane-1,12-diyl)bis(cyclopropane-1-carboxamide)(NP-5033), derivative, prodrug, or salt is administered between 500 and 1,000 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 60-100 mg orally per day and 1,1′-(dodecane-1,12-diyl)bis(cyclopropane-1-carboxamide)(NP-5033), derivative, prodrug, or salt is administered between 500 and 2,000 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 60-100 mg orally per day and 1,1′-(dodecane-1,12-diyl)bis(cyclopropane-1-carboxamide)(NP-5033), derivative, prodrug, or salt is administered between 1,000 and 2,000 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 60-100 mg orally per day and 1,1′-(dodecane-1,12-diyl)bis(cyclopropane-1-carboxamide)(NP-5033), derivative, prodrug, or salt is administered between 1,500 and 2,000 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 60-100 mg orally per day and 1,1′-(dodecane-1,12-diyl)bis(cyclopropane-1-carboxamide)(NP-5033), derivative, prodrug, or salt is administered between 1,000 and 3,000 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 60-100 mg orally per day and 1,1′-(dodecane-1,12-diyl)bis(cyclopropane-1-carboxamide)(NP-5033), derivative, prodrug, or salt is administered between 2,000 and 3,000 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 60-100 mg orally per day and 1,1′-(dodecane-1,12-diyl)bis(cyclopropane-1-carboxamide)(NP-5033), derivative, prodrug, or salt is administered between 10 and 500 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 60-100 mg orally per day and 1,1′-(dodecane-1,12-diyl)bis(cyclopropane-1-carboxamide)(NP-5033), derivative, prodrug, or salt is administered between 10 and 100 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 60-100 mg orally per day and 1,1′-(dodecane-1,12-diyl)bis(cyclopropane-1-carboxamide)(NP-5033), derivative, prodrug, or salt is administered between 100 and 200 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 60-100 mg orally per day and 1,1′-(dodecane-1,12-diyl)bis(cyclopropane-1-carboxamide)(NP-5033), derivative, prodrug, or salt is administered between 200 and 300 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 60-100 mg orally per day and 1,1′-(dodecane-1,12-diyl)bis(cyclopropane-1-carboxamide)(NP-5033), derivative, prodrug, or salt is administered between 300 and 400 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 30-100 mg orally per day and 1,1′-(dodecane-1,12-diyl)bis(cyclopropane-1-carboxamide)(NP-5033), derivative, prodrug, or salt thereof is administered between 10 and 3,000 mg per day or more.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 30-100 mg orally per day and 1,1′-(dodecane-1,12-diyl)bis(cyclopropane-1-carboxamide)(NP-5033), derivative, prodrug, or salt thereof is administered between 10 and 1,000 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 30-100 mg orally per day and 1,1′-(dodecane-1,12-diyl)bis(cyclopropane-1-carboxamide)(NP-5033), derivative, prodrug, or salt thereof is administered between 200 and 1,000 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 30-100 mg orally per day and 1,1′-(dodecane-1,12-diyl)bis(cyclopropane-1-carboxamide)(NP-5033), derivative, prodrug, or salt is administered between 500 and 1,000 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 30-100 mg orally per day and 1,1′-(dodecane-1,12-diyl)bis(cyclopropane-1-carboxamide)(NP-5033), derivative, prodrug, or salt is administered between 500 and 2,000 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 30-100 mg orally per day and 1,1′-(dodecane-1,12-diyl)bis(cyclopropane-1-carboxamide)(NP-5033), derivative, prodrug, or salt is administered between 1,000 and 2,000 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 30-100 mg orally per day and 1,1′-(dodecane-1,12-diyl)bis(cyclopropane-1-carboxamide)(NP-5033), derivative, prodrug, or salt is administered between 1,500 and 2,000 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 30-100 mg orally per day and 1,1′-(dodecane-1,12-diyl)bis(cyclopropane-1-carboxamide)(NP-5033), derivative, prodrug, or salt is administered between 1,000 and 3,000 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 30-100 mg orally per day and 1,1′-(dodecane-1,12-diyl)bis(cyclopropane-1-carboxamide)(NP-5033), derivative, prodrug, or salt is administered between 2,000 and 3,000 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 30-100 mg orally per day and 1,1′-(dodecane-1,12-diyl)bis(cyclopropane-1-carboxamide)(NP-5033), derivative, prodrug, or salt is administered between 10 and 500 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 30-100 mg orally per day and 1,1′-(dodecane-1,12-diyl)bis(cyclopropane-1-carboxamide)(NP-5033), derivative, prodrug, or salt is administered between 10 and 100 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 30-100 mg orally per day and 1,1′-(dodecane-1,12-diyl)bis(cyclopropane-1-carboxamide)(NP-5033), derivative, prodrug, or salt is administered between 100 and 200 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 30-100 mg orally per day and 1,1′-(dodecane-1,12-diyl)bis(cyclopropane-1-carboxamide)(NP-5033), derivative, prodrug, or salt is administered between 200 and 300 mg per day.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is administered in an amount of 30-100 mg orally per day and 1,1′-(dodecane-1,12-diyl)bis(cyclopropane-1-carboxamide)(NP-5033), derivative, prodrug, or salt is administered between 300 and 400 mg per day.

In certain embodiments, this disclosure relates to methods of improving urine concentrating ability in a subject receiving a vasopressin receptor antagonist therapy comprising administering N-(4-(7-chloro-5-hydroxy-2,3,4,5-tetrahydro-1H-benzo[b]azepine-1-carbonyl)-3-methylphenyl)-2-methylbenzamide (tolvaptan) to a human subject in combination with an effective amount of 1,1′-(dodecane-1,12-diyl)bis(cyclopropane-1-carboxamide)(NP-5033), derivative, prodrug, or salt thereof.

In certain embodiments, this disclosure relates to a combination of a vasopressin receptor antagonist and an activator of adenosine monophosphate activated protein kinase (AMPK) for use in use in methods disclosed herein.

In certain embodiments, this disclosure relates to the production of a medicament for use in methods disclosed herein comprising a vasopressin receptor antagonist and an activator of adenosine monophosphate activated protein kinase (AMPK).

In certain embodiments, this disclosure relates to a combination of a vasopressin receptor antagonist and an activator of adenosine monophosphate activated protein kinase (AMPK) for use in the treatment kidney function decline and preventing polyuria.

In certain embodiments, this disclosure relates to a combination of a vasopressin receptor antagonist and an activator of adenosine monophosphate activated protein kinase (AMPK) for use in use in the treatment of hyponatremia preventing polyuria.

In certain embodiments, this disclosure relates to the production of a medicament for use in the treatment of kidney function decline preventing polyuria comprising a vasopressin receptor antagonist and an activator of adenosine monophosphate activated protein kinase (AMPK).

### Pharmaceutical Compositions

In certain embodiments, this disclosure relates to pharmaceutical compositions comprising a vasopressin receptor antagonist and an activator of adenosine monophosphate activated protein kinase (AMPK) and a pharmaceutically acceptable excipient.

In certain embodiments, the pharmaceutically acceptable excipient is a carrier, solvent, diluent, coating, antibacterial or antifungal agent, or absorption delaying agent. In certain embodiments, the pharmaceutically acceptable excipient is selected from a diluent, disintegrant, solubilizing agent, or a lubricant.

In certain embodiments, the vasopressin receptor antagonist is selected from N-(4-(7-chloro-5-hydroxy-2,3,4,5-tetrahydro-1H-benzo[b]azepine-1-carbonyl)-3-methylphenyl)-2-methylbenzamide (tolvaptan), conivaptan, lixivaptan, and satavaptan, prodrug, derivative, or salt thereof.

In certain embodiments, the vasopressin receptor antagonist is N-(4-(7-chloro-5-hydroxy-2,3,4,5-tetrahydro-1H-benzo[b]azepine-1-carbonyl)-3-methylphenyl)-2-methylbenzamide (tolvaptan) and the activator of adenosine monophosphate activated protein kinase (AMPK)1,1′-(dodecane-1,12-diyl)bis(cyclopropane-1-carboxamide)(NP-5033), derivative, prodrug, or salt thereof.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 60-100 mg and 1,1′-(dodecane-1,12-diyl)bis(cyclopropane-1-carboxamide)(NP-5033), derivative, prodrug, or salt thereof is between 10 and 3,000 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 60-100 mg and 1,1′-(dodecane-1,12-diyl)bis(cyclopropane-1-carboxamide)(NP-5033), derivative, prodrug, or salt thereof is between 10 and 3,000 mg or more.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 60-100 mg and 1,1′-(dodecane-1,12-diyl)bis(cyclopropane-1-carboxamide)(NP-5033), derivative, prodrug, or salt thereof is between 10 and 1,000 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 60-100 mg and 1,1′-(dodecane-1,12-diyl)bis(cyclopropane-1-carboxamide)(NP-5033), derivative, prodrug, or salt thereof is between 200 and 1,000 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 60-100 mg and 1,1′-(dodecane-1,12-diyl)bis(cyclopropane-1-carboxamide)(NP-5033), derivative, prodrug, or salt is between 500 and 1,000 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 60-100 mg and 1,1′-(dodecane-1,12-diyl)bis(cyclopropane-1-carboxamide)(NP-5033), derivative, prodrug, or salt is between 500 and 2,000 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 60-100 mg and 1,1′-(dodecane-1,12-diyl)bis(cyclopropane-1-carboxamide)(NP-5033), derivative, prodrug, or salt is between 1,000 and 2,000 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 60-100 mg and 1,1′-(dodecane-1,12-diyl)bis(cyclopropane-1-carboxamide)(NP-5033), derivative, prodrug, or salt is between 1,500 and 2,000 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 60-100 mg and 1,1′-(dodecane-1,12-diyl)bis(cyclopropane-1-carboxamide)(NP-5033), derivative, prodrug, or salt is between 1,000 and 3,000 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 60-100 mg and 1,1′-(dodecane-1,12-diyl)bis(cyclopropane-1-carboxamide)(NP-5033), derivative, prodrug, or salt is between 2,000 and 3,000 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 60-100 mg and 1,1′-(dodecane-1,12-diyl)bis(cyclopropane-1-carboxamide)(NP-5033), derivative, prodrug, or salt is a between 10 and 500 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 60-100 mg and 1,1′-(dodecane-1,12-diyl)bis(cyclopropane-1-carboxamide)(NP-5033), derivative, prodrug, or salt is administered between 10 and 100 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 60-100 mg and 1,1′-(dodecane-1,12-diyl)bis(cyclopropane-1-carboxamide)(NP-5033), derivative, prodrug, or salt is between 100 and 200 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 60-100 mg and 1,1′-(dodecane-1,12-diyl)bis(cyclopropane-1-carboxamide)(NP-5033), derivative, prodrug, or salt is between 200 and 300 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 60-100 mg and 1,1′-(dodecane-1,12-diyl)bis(cyclopropane-1-carboxamide)(NP-5033), derivative, prodrug, or salt is between 300 and 400 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 30-100 mg and 1,1′-(dodecane-1,12-diyl)bis(cyclopropane-1-carboxamide)(NP-5033), derivative, prodrug, or salt thereof is between 10 and 3,000 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 30-100 mg and 1,1′-(dodecane-1,12-diyl)bis(cyclopropane-1-carboxamide)(NP-5033), derivative, prodrug, or salt thereof is between 10 and 3,000 mg or more.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 30-100 mg and 1,1′-(dodecane-1,12-diyl)bis(cyclopropane-1-carboxamide)(NP-5033), derivative, prodrug, or salt thereof is between 10 and 1,000 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 30-100 mg and 1,1′-(dodecane-1,12-diyl)bis(cyclopropane-1-carboxamide)(NP-5033), derivative, prodrug, or salt thereof is between 200 and 1,000 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 30-100 mg and 1,1′-(dodecane-1,12-diyl)bis(cyclopropane-1-carboxamide)(NP-5033), derivative, prodrug, or salt is between 500 and 1,000 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 30-100 mg and 1,1′-(dodecane-1,12-diyl)bis(cyclopropane-1-carboxamide)(NP-5033), derivative, prodrug, or salt is between 500 and 2,000 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 30-100 mg and 1,1′-(dodecane-1,12-diyl)bis(cyclopropane-1-carboxamide)(NP-5033), derivative, prodrug, or salt is between 1,000 and 2,000 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 30-100 mg and 1,1′-(dodecane-1,12-diyl)bis(cyclopropane-1-carboxamide)(NP-5033), derivative, prodrug, or salt is between 1,500 and 2,000 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 30-100 mg and 1,1′-(dodecane-1,12-diyl)bis(cyclopropane-1-carboxamide)(NP-5033), derivative, prodrug, or salt is between 1,000 and 3,000 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 30-100 mg and 1,1′-(dodecane-1,12-diyl)bis(cyclopropane-1-carboxamide)(NP-5033), derivative, prodrug, or salt is between 2,000 and 3,000 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 30-100 mg and 1,1′-(dodecane-1,12-diyl)bis(cyclopropane-1-carboxamide)(NP-5033), derivative, prodrug, or salt is a between 10 and 500 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 30-100 mg and 1,1′-(dodecane-1,12-diyl)bis(cyclopropane-1-carboxamide)(NP-5033), derivative, prodrug, or salt is administered between 10 and 100 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 30-100 mg and 1,1′-(dodecane-1,12-diyl)bis(cyclopropane-1-carboxamide)(NP-5033), derivative, prodrug, or salt is between 100 and 200 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 30-100 mg and 1,1′-(dodecane-1,12-diyl)bis(cyclopropane-1-carboxamide)(NP-5033), derivative, prodrug, or salt is between 200 and 300 mg.

In certain embodiments, N-(4-(7-chloro-5-hydroxy-2,3,4,5-tetrahydro-1H-benzo[b]azepine-1-carbonyl)-3-methylphenyl)-2-methylbenzamide (tolvaptan) is in an amount of 30-100 mg and 1,1′-(dodecane-1,12-diyl)bis(cyclopropane-1-carboxamide)(NP-5033), derivative, prodrug, or salt is between 300 and 400 mg.

In certain embodiments, the pharmaceutically acceptable excipient is a diluent. Examples include microcrystalline cellulose, other diluents may be, for example: calcium carbonate, calcium phosphate, calcium sulfate, cellulose acetate, erythritol, ethylcellulose, fructose, inulin, isomalt, lactitol, lactose, magnesium carbonate, magnesium oxide, maltitol, maltodextrin, maltose, mannitol, polydextrose, polyethylene glycol, pullulan, simethicone, sodium bicarbonate, sodium carbonate, sodium chloride, sorbitol, starch, sucrose, trehalose and xylitol.

In certain embodiments, the pharmaceutically acceptable excipient is a disintegrant.

Examples of a disintegrant may be, for example: alginic acid, calcium alginate, carboxymethylcellulose calcium, chitosan, colloidal silicon dioxide, croscarmellose sodium, crospovidone, glycine, guar gum, hydroxypropyl cellulose, low-substituted hydroxypropyl cellulose, magnesium aluminum silicate, methylcellulose, povidone, sodium alginate, sodium carboxymethylcellulose, sodium starch glycolate and starch.

In certain embodiments, the pharmaceutically acceptable excipient is a solubilizing agent. Examples of a solubilizing agent may be, for example: benzalkonium chloride, benzyl benzoate, sulfobutyl ether β-cyclodextrin sodium, cetylpyridinium chloride, cyclodextrins, diethylene glycol monoethyl ether, fumaric acid, hydroxypropyl beta cyclodextrin, hypromellose, lanolin alcohols, lecithin, oleyl alcohol, phospholipids, poloxamer, polyoxyethylene alkyl ethers, polyoxyethylene castor oil derivatives, polyoxyethylene sorbitan fatty acid esters, polyoxyl hydroxystearate, polyoxylglycerides, povidone, pyrrolidone, sodium lauryl sulfate, sorbitan esters (sorbitan fatty acid esters), tricaprylin, triolein and vitamin E polyethylene glycol succinate.

In certain embodiments, the pharmaceutically acceptable excipient is a lubricant. Examples of a lubricant may be, for example calcium stearate, glyceryl behenate, glyceryl dibehenate, glyceryl monostearate, glyceryl palmitostearate, a mixture of behenate esters of glycerin (e.g. a mixture of glyceryl dibehenate, tribehenin and glyceryl behenate), leucine, magnesium stearate, myristic acid, palmitic acid, poloxamer, polyethylene glycol, potassium benzoate, sodium benzoate, sodium lauryl sulfate, sodium stearate, sodium stearyl fumarate, stearic acid, talc, tribehenin and zinc stearate.

In certain embodiments, the pharmaceutically acceptable excipient is selected from lactose, sucrose, mannitol, triethyl citrate, dextrose, cellulose, methyl cellulose, ethyl cellulose, hydroxyl propyl cellulose, hydroxypropyl methylcellulose, carboxymethylcellulose, croscarmellose sodium, polyvinyl N-pyrrolidone, crospovidone, ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

In certain embodiments, the pharmaceutical composition is in the form of a tablet, pill, capsule, gel, gel capsule or cream. In certain embodiments, the pharmaceutical composition is in the form of a sterilized pH buffered aqueous salt solution or a saline phosphate buffer between a pH of 6 to 8, optionally comprising a saccharide or polysaccharide.

In certain embodiments, an agent disclosed herein may be used in the “free base form” or as a pharmaceutically acceptable salt, or as any mixture thereof. In one embodiment the agent is in the free base form. It is understood that “free base form” refers to the case where the agent is not in the form of a salt.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange.

Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

### Use of Activators of Adenosine Monophosphate Activated Protein Kinase (AMPK) to Lessen the Polyuric Side-Effect of Vasopressin Receptor Inhibitor Therapy for Autosomal Dominant Polycystic Kidney Disease (ADPKD)

Treatment with tolvaptan, which inhibits the type 2 vasopressin receptor (V2R), slows the progression of renal cyst development in autosomal dominant polycystic kidney disease (ADPKD). An unfortunate side effect of tolvaptan treatment is development of the need to urinate excessively, i.e., overt polyuria, greater than 2.5 L or 3 L over 24 hours in adults. This can result in non-compliance by some patients, thereby reducing the beneficial effect of tolvaptan treatment.

Tolvaptan is thought to improve ADPKD by decreasing cAMP production. Inhibiting the V2R results in polyuria and a form of nephrogenic diabetes insipidus. AMPK activation reduces polyuria in rats and mice with nephrogenic diabetes insipidus. Thus, experiments were developed to determine whether treatment with an AMPK activator could improve the polyuria in V2R inhibitor treated ADPKD patients without diminishing the beneficial effects.

Experiments are performed to determine whether AMPK activators can reduce polyuria in tolvaptan-treated mice with ADPKD without reducing the beneficial effect of tolvaptan on slowing cystogenesis. If the beneficial effect of tolvaptan is maintained but polyuria is reduced, this would improve quality of life for ADPKD patients and improve compliance with tolvaptan therapy.

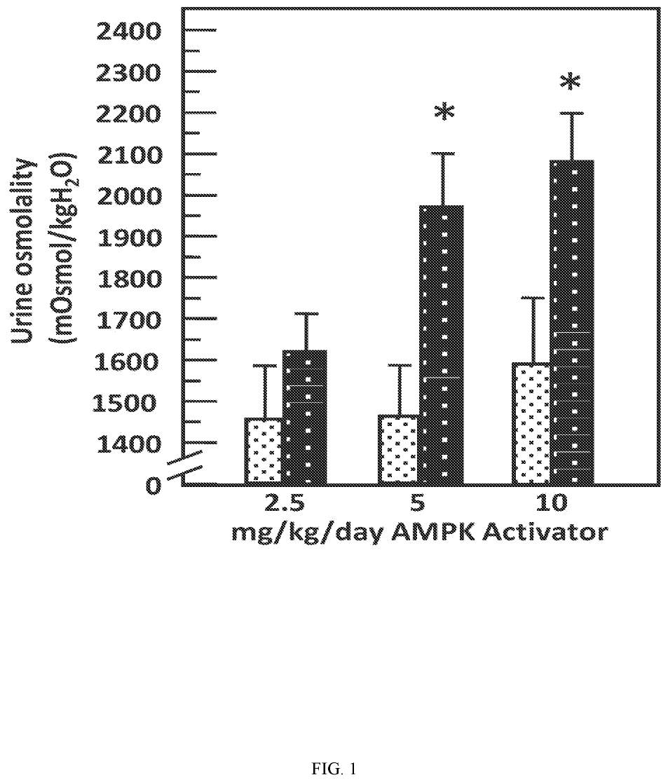

Data using NP-5033 shows improvement in urine concentrating ability in tolvaptan treated rats (see FIG. 1) and a vasopressin type 2 receptor knockout mouse model. Data indicates this AMPK activator increases urine osmolality. Preliminary data indicates that NP-5033 is potent, specific and does not cause hypoglycemia. Some AMPK activators, such as metformin, present a risk of developing hypoglycemia.

### Studies in Mice

One provides the AMPK activator agent to genetically modified mice (Pkd1^{R3277C/R3277C} hereinafter “ADPKD mice”) by gavage feeding, lx daily at a dose of 10 mg/kg/day. See Hoppe et al., J Am Soc Nephrol, 2015, 26(1):39-47. One solubilizes the agent in 40% (v/v) labrasol, 40% (v/v) labrafil and 20% transcutol (v/v) (4/4/2). One adds the agent to the lipid solution at 60° C. for 5-10 minutes to facilitate solubilization and thereafter cools to room temperature before feeding.

One uses two arms of mice in the study: 1) one administers the agent to tolvaptan ADPKD mice; and 2) control ADPKD mice receiving lipid solution without tolvaptan. One provides tolvaptan in formulated rodent chow that contains 0.1% tolvaptan ad libitum. One provides the AMPK activator daily oral gavage at a dose of 10 mg/kg/day. The volume containing the drug varies according to the animal weight at approximately 200 microliters. These mice develop cysts at 3-6 months.

### Experimental Procedures

On day 1, mice are age and weight matched and sorted into groups. Spot urines are taken for basal urine osmolality. The regular mouse chow will be replaced with tolvaptan laced chow for all 20 mice in this arm of the study.

On day 2, gavage feeding of AMPK activator begins with the 10 mice in the treatment group (designated TAA group). Control mice (receiving tolvaptan) receive the vehicle by oral gavage (designated TV group). This will continue daily until the animals are euthanized at the end of the experiment.

On day 4, 5 mice in the TV group and 5 mice from the TAA group are placed metabolic cages, urine is collected overnight. Mice will then be returned to their static cages.

On day 11, the remaining 5 mice in the TV and TAA groups are placed in metabolic cages and urine is collected overnight and volume noted. Mice are returned to their static cages.

This is repeated on alternating weeks through week 12.

Urine is analyzed for osmolality, Na, K, Cl content, and urinalysis by dip stick for protein, glucose, and blood. Renal ultrasound is performed on the anesthetized mice at weeks 3, 6 and 9.

At week 12 mice are euthanized by cervical dislocation and exsanguination. Blood will be collected by heart puncture. Kidneys are removed: 1 kidney are placed in 4% paraformaldehyde overnight in preparation for paraffin embedding for histology; the second kidney are freeze clamped, frozen in liquid N₂ and kept at −80° C. for future analysis. After the first group of 20 mice are euthanized, the next arm of the study is begun. Group 3, designated as CAA, are 10 ADPKD mice that do not receive tolvaptan treatment but are given daily gavage feeding of the AMPK activator. Group 4, designated as CV, will have 10 ADPKD that do not receive tolvaptan, but are given daily gavage feeding of the vehicle. These 2 groups are managed with the same treatments and timing as Groups 1 and 2, except for the regular chow. They are euthanized at week 12 or whichever week matches the endpoint for the first arm of the experimental protocol.

What is claimed is:

1. A method of treating or preventing polyuria as a side effect of a vasopressin receptor antagonist therapy comprising administering a vasopressin receptor antagonist to a human subject in combination with an effective amount of an activator of adenosine monophosphate activated protein kinase (AMPK).

2. The method of claim 1, wherein the subject is diagnosed with autosomal dominant polycystic kidney disease (ADPKD).

3. The method of claim 1, wherein the subject is diagnosed with polyuria.

4. The method of claim 1, wherein that vasopressin receptor antagonist is tolvaptan.

5. The method of claim 1, wherein the AMPK activator is 1,1′-(dodecane-1,12-diyl)bis(cyclopropane-1-carboxamide)(NP-5033) or salt thereof.

6. A method of treating or preventing polyuria as a side effect of a vasopressin receptor antagonist therapy comprising administering tolvaptan to a human subject in combination with an effective amount of 1,1′-(dodecane-1,12-diyl)bis(cyclopropane-1-carboxamide)(NP-5033) or salt thereof.

7. The method of claim 6, wherein tolvaptan is administered in an amount of 30-100 mg orally per day and 1,1′-(dodecane-1,12-diyl)bis(cyclopropane-1-carboxamide)(NP-5033) or salt thereof is administered between 10 and 3,000 mg per day.

8. A method of treating autosomal dominant polycystic kidney disease (ADPKD) comprising administering tolvaptan to a human subject in combination with an effective amount of 1,1′-(dodecane-1,12-diyl)bis(cyclopropane-1-carboxamide)(NP-5033) or salt thereof.

9. The method of claim 8, wherein tolvaptan is administered in an amount of 60-100 mg orally per day and 1,1′-(dodecane-1,12-diyl)bis(cyclopropane-1-carboxamide)(NP-5033) or salt thereof is administered between 10 and 3,000 mg per day.

10. A pharmaceutical composition comprising a vasopressin receptor antagonist and an activator of adenosine monophosphate activated protein kinase (AMPK) and a pharmaceutically acceptable excipient.

11. The pharmaceutical composition of claim 10, wherein that vasopressin receptor antagonist is tolvaptan.

12. The pharmaceutical composition of claim 10, wherein the AMPK activator is 1,1′-(dodecane-1,12-diyl)bis(cyclopropane-1-carboxamide)(NP-5033) or salt thereof.

13-15. (canceled)
